(19) United States
(12) Patent Application Publication
(10) Pub. No.: US 2024/0424210 A1
(43) Pub. Date: Dec. 26, 2024

(54) PERSONALIZED SIMPLIFIED MEAL COMPENSATION

(71) Applicant: INSULET CORPORATION, Acton, MA (US)

(72) Inventors: Mert SEVIL, Manchester, NH (US); Yibin ZHENG, Hartland, WI (US); William WHITELEY, Knoxville, TN (US); Rangarajan NARAYANASWAMI, Weston, MA (US); Saeed SALAVATI, Sugar Land, TX (US); Sam CARL, Acton, MA (US)

(21) Appl. No.: 18/749,512

(22) Filed: Jun. 20, 2024

Related U.S. Application Data
(60) Provisional application No. 63/509,843, filed on Jun. 23, 2023.

(51) Int. Cl. A61M 5/172; G16H 20/17

(52) U.S. Cl. CPC A61M 5/1723; G16H 20/17; A61M 2230/201

(57) ABSTRACT

Disclosed are a drug delivery system, a drug delivery device, a controller and a number of techniques to personalize the delivery of a bolus dosage. In an example, a drug delivery system may include a processor and a memory. The memory may be operable to store instructions that, when executed by the processor, cause the processor to receive a request to deliver a bolus dosage, retrieve a history of glucose measurement values, extract features from the history of glucose measurement values, assign a value to extracted features and calculate a personalized factor, and based on the personalized factor or factors, modify a calculation for an amount of a medicament to deliver. Additionally or alternatively, the personalized factor may be used to modify parameter settings of a medicament delivery algorithm. The described systems may track a user's personalized factor over a period of time.

### CROSS-REFERENCE TO RELATED APPLICATIONS

This application claims priority to and the benefit of U.S. Provisional Application No. 63/509,843, filed Jun. 23, 2023, the entirety of which is incorporated herein by reference.

### BACKGROUND

A conventional drug delivery system may include, for example, wearable medicament delivery devices. The wearable medicament delivery device can be designed to deliver any type of liquid drug to a user. In specific embodiments, the drug delivery device can be, for example, an OmniPod® drug delivery device manufactured by Insulet Corporation of Acton, Massachusetts. The drug delivery device can be a device such as those described in U.S. Pat. Nos. 7,303,549, 7,137,964, or U.S. Pat. No. 6,740,059, each of which is incorporated herein by reference in its entirety.

Patients with diabetes suffer unneeded anxiety as they strive to keep their blood sugar under control especially in response to changes to diet, activity, situations, or the like. One of the primary changes is meal consumption and the delivery of a meal-compensating bolus. However, often times a user does not know or consider the effects of insulin or equivalent drugs that were previously delivered or any changes to how sensitive their body is to insulin at the present time.

Automated drug delivery systems are algorithm-driven, which helps maintain a user's glucose in a target range but parameter settings of the algorithm are often default settings that are not specific to the individual user. One significant and common tuning parameter of these controllers is the target glucose setpoint. Each individual user has a different setpoint and each individual user typically determines their set-point themselves. A typical set point range may be 110 mg/dl to 150 mg/dl, for example. The target glucose setpoint or setpoint is a sensitive parameter and needs to be selected accurately. A high setpoint can result in hyperglycemia, while a low setpoint can result in hypoglycemia. Optimal selection of a target glucose setpoint is a difficult task for users, which is a burden for them. Inaccurate selection of the target set point may result in poor glycemic outcomes for the user. Moreover, the physiology of individuals can change over-time, therefore fixed and non-adaptive setpoints may not perform perfectly under all circumstances. For example, if someone's drug sensitivity (e.g., insulin sensitivity) changes due to hormonal factors, the setpoint should also be adaptive to these changes.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one aspect, a drug delivery system is provided. The drug delivery system may include a memory and a processor. The memory may be configured to store programming code and data. The processor may be coupled to the memory and operable to execute the programming code. The processor, when executing the programming code, is operable to receive a plurality of glucose measurement value. A plurality of features are extracted from the received plurality of glucose measurement value. The processor evaluates the plurality of extracted features, and, based on a result of the evaluation, applies a respective weighting to each of the plurality of extracted features. The processor may derive a personalized factor based on the respective weighting of each of the plurality of extracted features, and utilize the personalized factor to calculate a bolus dosage to be delivered to a user.

In a further aspect, a drug delivery system includes a memory and a processor. The memory may be operable to store a medicament delivery algorithm. The medicament delivery algorithm, when executed by the processor, may cause the processor to receive a meal announcement. In response to the meal announcement, the processor may retrieve a history of glucose measurement values. The processor may extract feature values from the history of glucose measurement values, and determine a weighted extracted feature value by applying a weight value to each respective extracted feature value. A personalized factor may be determined based on the weighted extracted feature values. The processor, based on the personalized factor, may modify a parameter setting of the medicament delivery algorithm.

In yet another aspect, a drug delivery system, includes a processor operable to execute programming instructions related to a medicament delivery algorithm. The drug delivery system also includes a memory operable to store the medicament delivery algorithm and data related to a user's analyte history, where the processor is operable to determine a first personalized factor for a first period of time, modify one or more parameter settings based on the first personalized factor, receive analyte measurement values after determining the first personalized factor, determine a second personalized factor for a second period of time, generate a tracking personalized factor based on the first personalized factor and the second personalized factor, and adjust based on the tracking personalized factor the modified one or more parameter settings, which were modified based on the first personalized factor.

### BRIEF DESCRIPTION OF THE DRAWINGS

To easily identify the discussion of any particular element or act, the most significant digit or digits in a reference number refer to the figure number in which that element is first introduced.

FIG. 1 illustrates an aspect of the subject matter in accordance with one embodiment.

FIG. 2 illustrates a flowchart of an exemplary personalized factor process as described herein.

FIG. 3 illustrates an exemplary presentation of the subject matter in accordance with one embodiment.

FIG. 4 illustrates an aspect of the subject matter in accordance with one embodiment.

FIG. 5 illustrates an aspect of the subject matter in accordance with one embodiment.

FIG. 6 illustrates a flowchart of an exemplary process that utilizes a personalized factor for setting parameters of a control application.

FIG. 7A illustrates an example for tracking a user's personalized factor during a period of time in accordance with at least one embodiment of the disclosed subject matter.

FIG. 7B illustrates an exemplary flowchart for tracking a user's personalized factor during a period of time in accordance with at least one embodiment of the disclosed subject matter.

FIG. 8 illustrates an exemplary drug delivery system suitable for implementing the processes and techniques as described herein.

### DETAILED DESCRIPTION

The simplified bolusing method described herein lessens the burden of meal compensation with an automated, algorithm-based medication delivery. The simplified bolus may be triggered by a meal announcement from the user and may include, in some examples, one or more components. For example, the one or more components may be an immediate bolus delivery and a constraint relaxation period. In other examples, the immediate bolus delivery and a constraint relaxation period may be separately implemented. Additional components may include a basal delivery, a total daily drug determination, or the like.

As part of determining the bolus dosage amount for the immediate bolus delivery and the constraint relaxation period, it would be helpful to be able to determine a user's medicament utilization. In order to determine an individuals' medicament utilization, a historical data-driven personalization algorithm may be implemented. Some embodiments of the personalization algorithm learns personalized factors over time from historical glycemic outcomes (e.g., glucose measurement value). As explained in further detail with reference to the figures, a personalized factor may show a user's tendency to hyperglycemia, hypoglycemia, or euglycemia. The personalized factor may, for example, be used to update an insulin dosage (i.e., a bolus dosage) where hyperglycemic users use more insulin, and hypoglycemic users need less insulin. Based on the personalized factor, the immediate bolus amount and constraint relaxation settings may be updated. As a result, the described examples provide the user with optimized glycemic performance by taking into account such personalized factors. In an example, the personalized factor enables the medicament delivery algorithm to operate safely by providing a conservative insulin dosage for people who are prone to hypoglycemia due to high insulin delivery for meal compensation. The described techniques also provide adaptivity. For example, as a user's history changes over time, the describe processes may be configured to learn changes from the historical data over time and adjust the insulin dosage and the constraint relaxation period.

Therefore, the determination of a personalized factor over time based on historical data and utilizing the personalized factor into simplified bolusing determinations would enhance the performance of an AID system. Since each individual patient with diabetes has different glucose-insulin dynamics, basal-bolus split and carbohydrate to insulin ratio, they may need different insulin dosages.

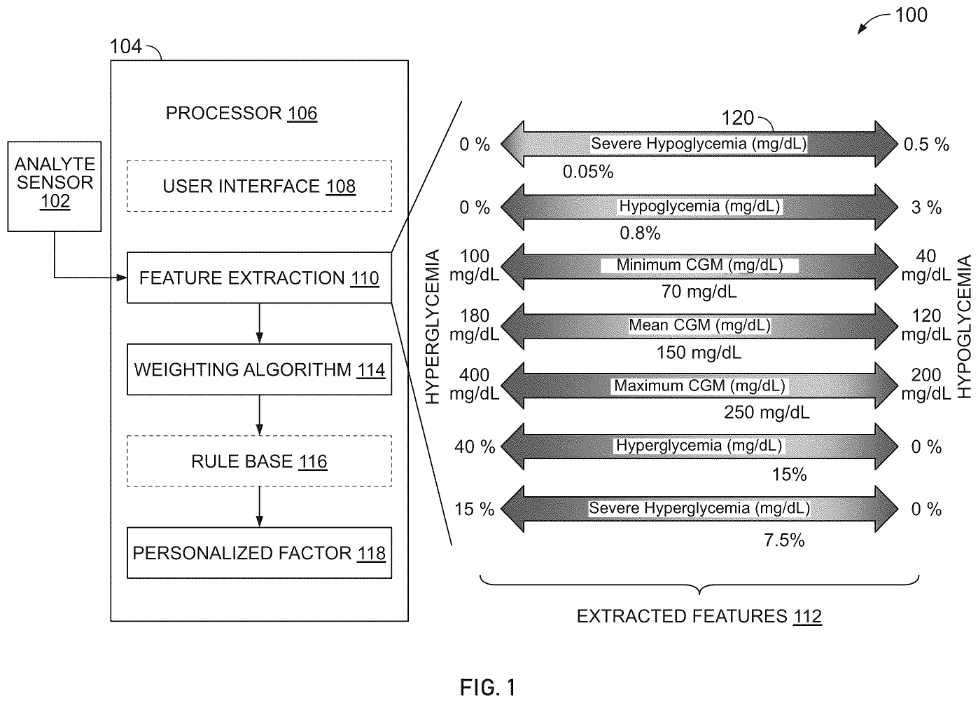

FIG. 1 illustrates a system that is configured to implement the techniques and processes described herein. For example, the system 100 may include an analyte sensor 102, and a controller or wearable medicament delivery device 104. In an example, the controller or wearable medicament delivery device 104 may include a processor 106, and a user interface 108 (which may be optional in the case of wearable medicament delivery device). The controller or wearable medicament delivery device 104 may have computer programming code stored in a memory that includes, for example, a feature extraction component 110, an extracted features 112, a weighting algorithm 114, a rule base 116, and a personalized factor generator 118. The processor may be operable to execute a medicament delivery algorithm (MDA).

The analyte sensor 102, which is described in more detail with respect to a later system example, is operable to provide analyte measurement values, such as glucose measurement values, ketone measurement values, combinations of analyte measurement values, or the like to the processor 106, and, in particular, to the feature extraction component 110. The feature extraction component 110 is operable to evaluate the provided analyte measurement values to extract features, such as extracted features 112. The extracted features 112 have a raw feature value. For example, extracted feature 120 may have a raw feature value of 0.05%, or the like. The raw feature value may be any format of value, such as a percentage, numbers, or the like. The number of extracted features 112 may range from 1, 1 to 7, 1-20, 1-25, 1-100, or the like, depending upon the desired granularity of the personalized factor. In the example of FIG. 1, the number of extracted features 112 is 7.

The weighting algorithm 114 may receive the values of the extracted features 112 and apply a weight to the respective extracted features of the number of extracted features 112. As explained in more detail with reference to other examples, the weighting algorithm 114 may process the raw feature value using one or more mathematical functions and/or techniques that normalize the respective extracted feature values (i.e., raw values) of the extracted features 112. The weighting algorithm 114 utilizing either weights or scores outputs a normalized extracted feature value that may be further processed.

The rule base 116 may be an optional process which applies a set of rules to the normalized extracted feature value. For example, the rule base 116 may group different extracted features values in different descriptive memberships, such as very low descending, very low ascending, medium descending or medium ascending. Each descriptive membership may have an individual value that is applied to the respective descriptive membership. In some instances, there may be more memberships than there are extracted feature values in which case extracted features may belong to multiple memberships.

Based on the output of the rule base 116, the personalized factor generator 118 may be operable to generate a single value that is representative of user's tendency for hyperglycemia, hypoglycemia, or euglycemia as well as indicating how well a user remains within range of their target glucose setpoint. The personalized factor, whether based on one extracted feature or based on several extracted features is useful in determining an immediate bolus amount and updating constraint relaxation settings of a medicament delivery algorithm (MDA) as well as other calculations and MDA setting updates.

The personalized factor generated by the personalized factor generator 118 may be envisioned as a value that on a common scale categorizes system users relative to the MDA's capability to control the user's diabetes. The processes executed by feature extraction component 110, weighting algorithm 114, rule base 116, if used, and personalized factor generator 118 are described in more detail with reference to the other examples.

In an example, a drug delivery device may be enabled to respond to a “one touch” bolus command that is input into a user interface and/or via an input-output device, such as a controller or a smart accessory device. A “one touch” bolus command may be actuation of a (digital) button of a user interface, which prompts the AID system to calculate a bolus dose for the user. In some embodiments, the AID system may automatically deliver the bolus dose after calculation, in other embodiments, an additional user intervention by the user is required to deliver the bolus after calculation. A controller, a wearable medicament delivery device, a smart accessory device or the like may be operable to receive, via a user interface, the “one touch” command. The “one touch” command may be interpreted by a processor as a request for a bolus. In response to the request for the bolus, the processor may perform a number processes.

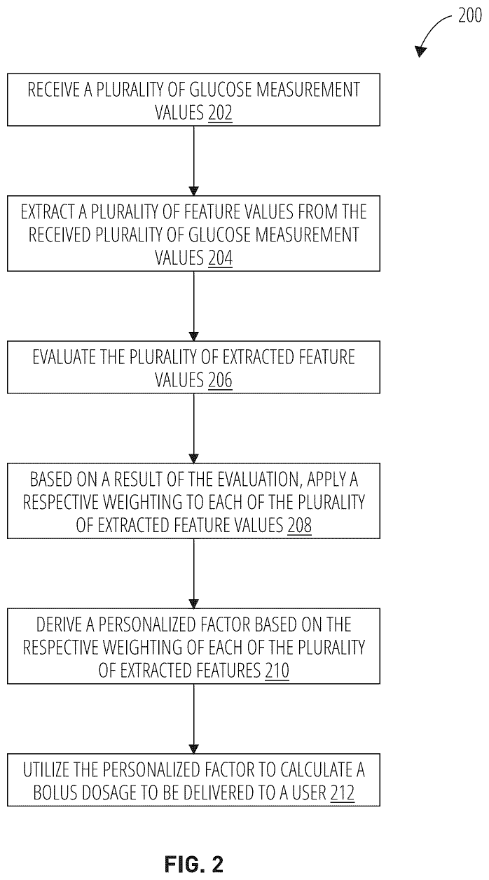

One of the number of processes performed by the processor may include the following process 200 discussed with reference to FIG. 2.

In step 202, the processor implementing the process 200 may receive a plurality of analyte measurement values. In this example, the analyte measurement values are glucose measurement values, but other analyte measurement values such as ketone measurement values, or the like may also be used. The glucose measurement value may be provided by an analyte sensor that is coupled to the user, is accessed by the user, or may be retrieved from memory or a cloud-based service. For example, the analyte sensor may be a continuous glucose monitor (CGM) affixed to the skin of a user that outputs a user's glucose measurement value (or data indicative of a glucose measurement value) and/or other data, such as a timestamp, trend information, previous glucose measurement value, and the like. The plurality of glucose measurement values may be used to form a CGM history of the user. The CGM history may include glucose measurement values obtained by the CGM over a 3-hour period, a 12-hour period, a 24-hour period, a 36-hour period, a 48-hour period, a period measured in days, weeks, or months, or the like. In some embodiments, the CGM history includes analyte measurement values, in particular glucose measurement values, obtained in a period of time between 3 hours to 1 month prior to the current point in time. The CGM history may be stored in a memory of one or more devices that are components of the drug delivery system shown in later examples as well as, or alternatively, in a cloud server that is part of or provides the cloud-based service (as shown in a later example).

In step 204, the process 200 may extract one or more features from the received plurality of glucose measurement values. In an example, the processor may extract a number of unique features from the CGM history. The number of unique features extracted from the CGM history may range from 1 or 2, or 3 to 10, 7, or the like. In some embodiments the number of unique features extracted from the CGM history is between 1 to 10. In one example, features extracted and/or derived by the processor from the data in the CGM history may include features such as severe hypoglycemia percentage (i.e., a percentage (%) of time of blood glucose (BG)<50 mg/dL), hypoglycemia percentage (i.e., a percentage (%) of % time of BG<70 mg/dL), a minimum glucose value (in, for example, mg/dL) from the CGM, a mean glucose value (mg/dL), a maximum glucose value (in, for example, mg/dL) from the CGM, a hyperglycemia percentage (i.e., a percentage (%) of time of BG>180 mg/dL), and a severe hyperglycemia percentage (i.e., a percentage (%) of time of BG>550 mg/dL). The threshold for severe hypoglycemia, hypoglycemia, hyperglycemia and severe hyperglycemia may differ from the numbers mentioned above. Each of the extracted features is given a raw extracted feature value. Of course, other features may be extracted from the data in the CGM history, such as a standard deviation of a glucose measurement value in the CGM history, a time-in-range with respect to a target glucose setpoint or range, a median glucose measurement value, time-related data with respect to different glucose measurement values (e.g., the average glucose measurement value received from the CGM between the hours of 8 am and 12 μm, for example), and the like. It is also envisioned that the glucose measurement values may be received from a blood glucose device that utilizes a finger-stick method and/or from a CGM.

As used herein, a “target glucose set point” may refer to a glucose control level setting or a controller glucose level setting, usually measured in mg/dL, of a medicament delivery algorithm. The medicament delivery algorithm may use the glucose level setting to establish a medicament delivery schedule that may include basal and bolus doses and that may be established ahead of time or contemporaneously with the determination of a number of items of data, including the determination of one or more personalized factors, ketone levels, glucose levels, and the like as described herein.

At step 206, process 200 evaluates the one or more extracted features. In an example, the processor, when evaluating the plurality of extracted features, is operable to assign a respective value (i.e., a raw extracted feature value) to each extracted feature of the one or more extracted features.

In step 208, based on a result of the evaluation, a respective weighting may be assigned to each of the one or more extracted features. For example, each extracted feature may have a respective value and the processor may be further operable to apply a weighting algorithm to the plurality of extracted features. Different weighting methodologies include fuzzy-logic approaches, iterative proportional fitting (also referred to as “raking”), matching, propensity weighting, or the like. The processor may obtain from the weighting algorithm, a respective weight value to be assigned to each extracted feature of the one or more extracted features.

In a specific example for weighting the respective extracted feature values, each extracted feature value may be used as an input to an artificial intelligence-based fuzzy-logic algorithm. The artificial intelligence-based fuzzy-logic algorithm may fuzzify the extracted feature values utilizing optimized fuzzification curves. Additionally, a rule base may utilize membership functions that produce membership values. For example, the rule base may be a set of rules (e.g., inequalities or the like that are applied to the extracted feature values), and the result of the rule may indicate the membership value. The membership values may be defuzzified utilizing a minimum-maximum methodology and may provide the respective weight value assigned to each extracted feature of the one or more extracted features.

In step 210, the computer instructions implementing the process 200 may cause a processor to derive a personalized factor based on the respective weighting of each of the one or more extracted features and the respective value of each of the respective extracted features. In an example, the processor may, after applying the respective weight values assigned to each respective extracted feature value of the one or more extracted features, normalize each weighted extracted feature value, determine an average value of the normalized respective weighted extracted feature values, and set the average value as the personalized factor. Numerical techniques other than averaging may be used to derive the personalized factor. The normalization may include adjusting the extracted feature values to a notionally common scale, in particular adjusting the measured extracted feature values to a normal distribution. As such, the personalized factor is a value obtained from the processor, for example, executing a summation of the average values that is scaled with 0-1 normalization, or a more sophisticated function.

In step 212, in response to the bolus request, the process 200 utilizes the personalized factor to calculate a bolus dosage to be delivered to a user. This calculated bolus dosage represents the value of medicament that is (to be) delivered to the user, for example, in response to the user pressing a simple meal announcement button, or a “one touch button” in conjunction with consumption of a meal or food by the user. Alternatively, or additionally, the calculated bolus dosage may represent a correction bolus that is intended to more quickly return the user's glucose values to within range of the user's target glucose setpoint.

As mentioned above, the personalized factor(s) may be used as an indicator of a user's glycemic risk. For example, the processor may determine a degree to which the respective weighted extracted features indicate a user bias or tendency toward hyperglycemia or hypoglycemia or euglycemia; and assign a value as the personalized factor based on the determined degree. In an example, a personalized factor lower than 0.5 suggests a risk or tendency for hypoglycemia of the user (with 0 indicating a severe hypoglycemic risk), while a personalized factor higher than 0.5 factor suggests a risk or tendency for hyperglycemia of the user (with 1 indicating a severe hyperglycemic risk), and a personalized factor that is equal to or approximately 0.5 indicates that the user's glucose levels tend to be euglycemic. The processor may utilize the personalized factor(s) for determining the risk direction and also the magnitude of the glycemic risk. For example, a user who has a personalized factor of 0.05 needs more attention due to the increased potential of the user experiencing a hypoglycemic event as well as quicker algorithmic action than a person who has a 0.45 personalized factor, which indicates a euglycemic user.

The calculated personalized factor(s) may be used to determine a bolus dosage for the user. A baseline bolus may be calculated using a user's total daily insulin (TDI) by multiplying the user's TDI by a baseline bolus coefficient. The baseline bolus coefficient may be approximately 0.06. In other words, a baseline bolus may be 6% of a TDI. Other values may be used (e.g., 0.04 to 0.18). Accordingly, in some embodiments, the baseline bonus may be between about 4% to about 18% of the TDI. The calculated personalized factor(s) may be used to adjust an immediate meal bolus dosage. For example, when the combined personalized factor is closer to 1 the user's bolus dosage may be increased and when the user's combined personalized factor is closer to 0 the user's bolus dosage may be decreased.

In a further example, the processor may calculate a medicament dosage, which may be a bolus dosage, or a bolus dosage adjustment, based on the combined personalized factor (PF) for a meal bolus using the following Equations 1 and 2:

$$One Touch Dose Change ( \% ) = ( P F - B B S P ) \times α , \qquad ( Equation 1 )$$

where α is an adaptation speed coefficient, PF is combined personalized factor, and the BBSP is a factor representing a basal/bolus split percentage of the total daily insulin (TDI). In some embodiments the BBSP may be between about 2 to about 0.5, more specifically 1.5 to 0.6 and in particular between about 1.2 to 0.8.

$${x}_{n e w} = ( x + One Touch Dose Change ) ( \% T D I ) + \qquad ( Equation 2 )$$

$$Safe Required IOB - Current Total IOB ,$$

where x_{new} is an updated meal dose TDI (%), x is a current meal dose TDI (%) (i.e. the current percentage of TDI regarded as suitable for the user as a meal dose), and the Safe Required IOB is a baseline amount of IOB that the user should have for safety, and Current Total IOB is the current total amount of IOB the user is predicted by the device to have. The basal-bolus split point (BBSP) may be set at different ranges for a different users depending upon a user's parameters, such as total daily insulin need, correction and/or sensitivity factors, insulin-to-carbohydrate ratio, and the like. An initial meal dose, i.e. a first current meal dose, may be a standard setting by the AID or set by a physician.

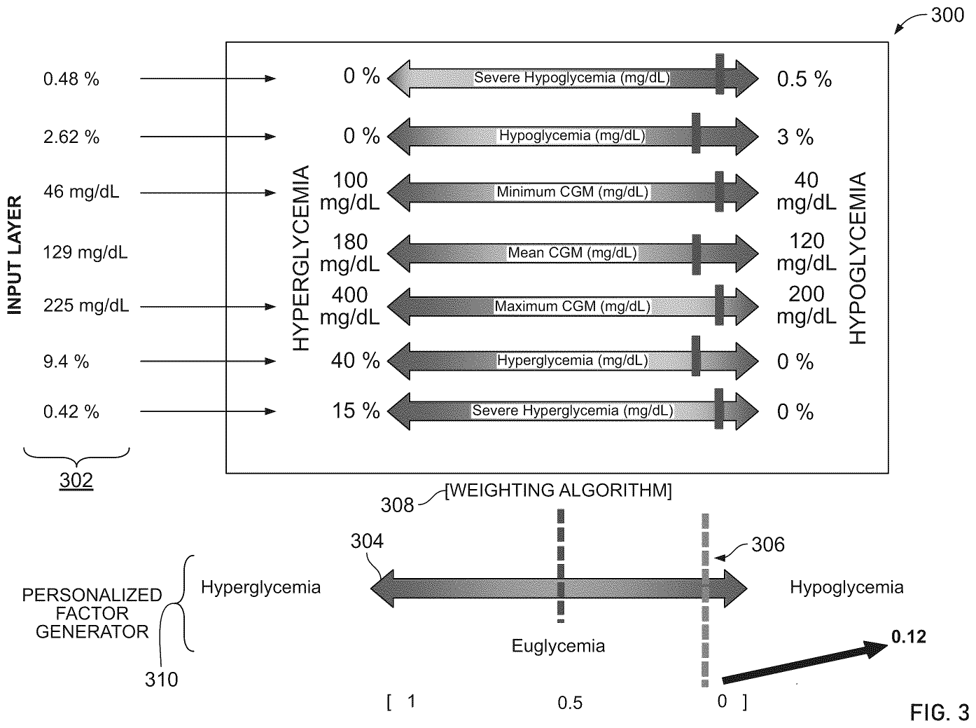

It may be beneficial to describe examples of the output of the process 200 with reference to different input values. FIG. 3 illustrates a graphic illustrating an exemplary output of the process 200.

In graphic 300 of FIG. 3, the raw extracted feature values 302 may be obtained by a feature extraction component, and be input for processing into a weighting algorithm 308. The weighting algorithm 308 may process the raw extracted feature values 302. For example, the weighting algorithm 308 may process the raw feature value using one or more mathematical functions and/or techniques, for example, a fuzzification scheme and/or a rule base, or the like, that normalizes the respective raw extracted feature values 302.

In the example of FIG. 3, the output from the weighting algorithm 308 is provided as an input into the personalized factor generator 310. The personalized factor generator 310 utilizes the personalized factor scale 304 to generate the personalized factor 306 based on the raw extracted feature values 302. In this example, the user's personalized factor is 0.12, which indicates that the user is close to consistently being hypoglycemic.

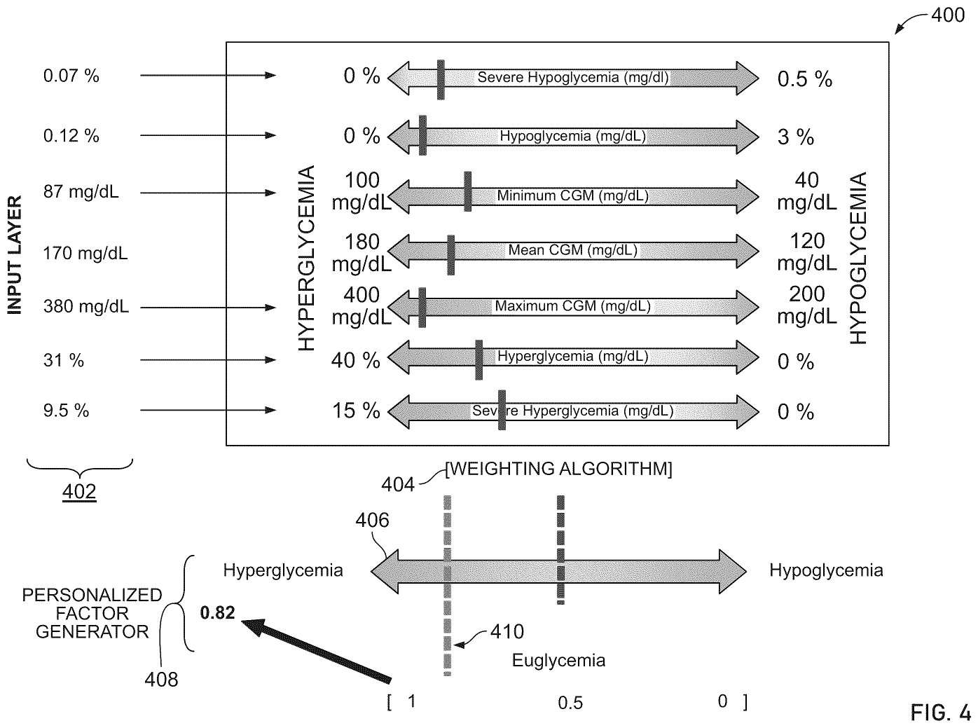

In another example, FIG. 4 illustrates a graphic illustrating another exemplary output of the process 200.

In graphic 400 of FIG. 4, the raw extracted feature values 402 may be obtained by a feature extraction component, and be input for processing into a weighting algorithm 404. The weighting algorithm 404 may process the raw extracted feature values 402. For example, the weighting algorithm 404 may process the raw feature value using one or more mathematical functions and/or techniques, for example, a fuzzification scheme and/or a rule base, or the like, that normalizes the respective raw extracted feature values 402.

In the example of FIG. 4, the output from the weighting algorithm 404 is provided as an input into the personalized factor generator 408. The personalized factor generator 408 utilizes the personalized factor scale 406 to generate the personalized factor 410 based on the raw extracted feature values 402. In this example, the user's personalized factor is 0.82, which indicates that the user is close to consistently being hyperglycemic.

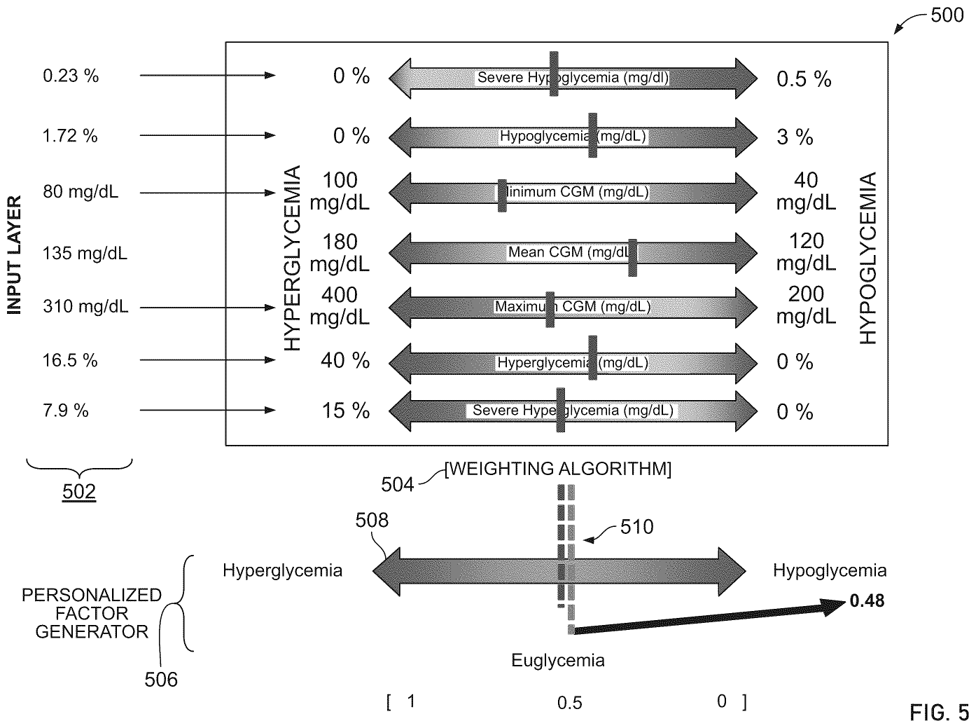

In another example, FIG. 5 illustrates a graphic illustrating yet another exemplary output of the process 200.

In graphic 500 of FIG. 5, the raw extracted feature values 502 may be obtained by a feature extraction component, and be input for processing into a weighting algorithm 504. The weighting algorithm 504 may process the raw extracted feature values 502. For example, the weighting algorithm 504 may process the raw feature value using one or more mathematical functions and/or techniques, for example, a fuzzification scheme and/or a rule base, or the like, that normalizes the respective raw extracted feature values 502.

In the example of FIG. 5, the output from the weighting algorithm 504 is provided as an input into the personalized factor generator 506. The personalized factor generator 506 utilizes the personalized factor scale 508 to generate the personalized factor 510 based on the raw extracted feature values 502. In this example, the user's personalized factor is 0.48, which indicates that the user is close to consistently being euglycemic.

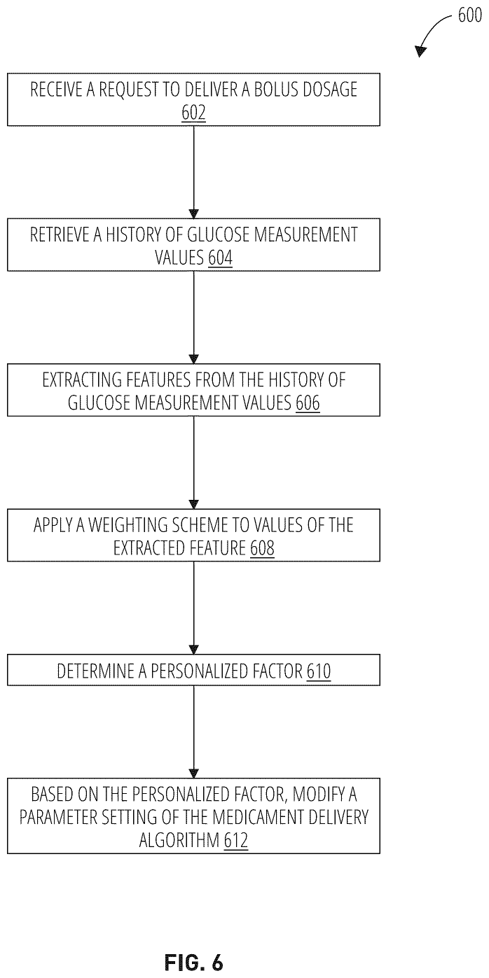

FIG. 6 illustrates a flowchart of an exemplary process that utilizes a combined personalized factor for setting parameters of a control application. The combined personalized factor may have additional uses including determining another component of the ADD algorithm, such as determining a setpoint adjustment based on the personalized factor. Some of the process 600 steps may be similar to those executed in process 200, therefore, a detailed discussion of those steps will not be made.

For example, in step 602, process 600 receives a request to deliver a bolus dosage. For example, a drug delivery device may be enabled to respond to a “one touch” bolus command that is input into a user interface and/or via an input-output device, such as a controller or a smart accessory device. A controller, a wearable medicament delivery device, a smart accessory device or the like may be operable to receive, via a user interface, the “one touch” command for delivery of a bolus dosage.

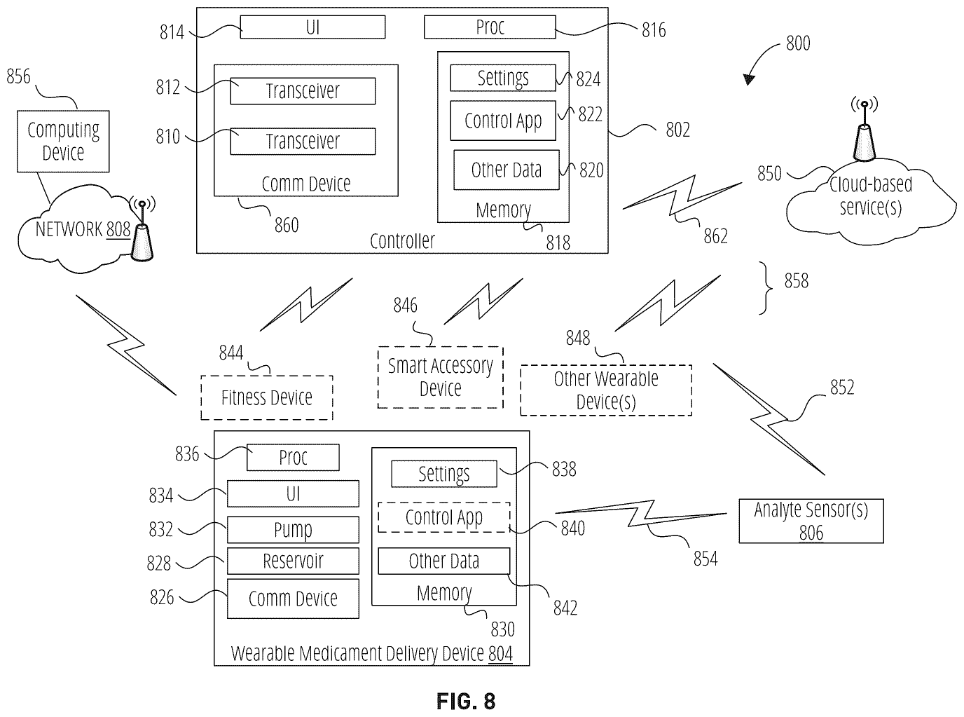

In response to the meal announcement, the process 600 retrieves a history of glucose measurement values in step 604. The history of glucose measurement values (also referred to as a glucose measurement value history) or CGM history may be stored in a memory of one or more devices that are components of the drug delivery system (described with reference to FIG. 8) as well as, or alternatively, in a cloud server that is part of the cloud-based service. For example, a processor may access a memory, a cloud server, or the like to obtain the glucose measurement value history or CGM history, which may be glucose measurement values obtained from a CGM over a 3 hour period, a 12 hour period, a 24 hour period, a 36 hour period, a 48 hour period, or the like.

In step 606, the process 600 causes the processor to extract features from the history of glucose measurement values. In step 208, the process 600 assigns a weighting scheme value to each of the extracted features. In step 610, the process 600 determines a combined personalized factor. Steps 606, 608 and 610 may be performed in substantially the same manner as described above with reference steps 204-210 of process 200.

In step 612, the process 600, based on the combined personalized factor, causes a processor to modify a parameter setting of medicament delivery algorithm. In the process 600, the processor after deriving the user's combined personalization factor may cause a modification of the user's bolus dosage calculation by adjusting one or more constraint settings. For example, the processor may modify or adjust constraint settings such as a one time constraint, an integral constraint, a drug-on-board (DOB) constraint, and/or a cost function.

Step 612 is an extension of the process 600 that utilizes one or more of the disclosed techniques to perform a number of different parameter adjustments. For example, the process 600 may enable a processor to make an adaptive adjustment, a personalized adjustment, and/or a dynamic adjustment to parameter settings of a medicament delivery algorithm (MDA). The respective adaptive adjustment, personalized adjustment and/or dynamic adjustment provide the benefits of increased glycemic performance of the MDA and an overall more optimal performance of the drug delivery system.

In an example of an adaptive adjustment, a processor executing the MDA may evaluate recent historical glucose measurement value data (e.g., data from the last 24, 48 or 72 hours) and may make modifications to one or more parameter settings based on recent glycemic outcomes. In the example of a medicament being insulin, if the user recently needs more insulin due to an insulin sensitivity change, the increased need for insulin is reflected as a recent glycemic outcome. In response, the described processes become aware of this recent glycemic outcome and are operable to make an appropriate target glucose setpoint adjustment.

In an example of a personalized adjustment, nearly all persons with diabetes have a different glycemic profile and the disclosed processes may utilize the personalized factor(s) or combined personalized factor to enable, for example, a gradual reduction of the user's target glucose setpoint if the particular user's glucose measurement values consistently tends towards hyperglycemia.

In yet a further example, the disclosed processes may be executed to enable a dynamic adjustment, which enables the algorithm, when a user has a change in lifestyle (such as, for example, starting a vigorous exercise regimen or a particular type of diet), to reduce the target glucose setpoint to reduce the risk of exercise-related or diet-related hypoglycemia.

Each of the above examples of a parameter change have been shown to improve glycemic performance of the drug delivery system (i.e., the percentage of time that the user's glucose measurement values were within range of their respective target glucose setpoint). Some results show that setpoint changes also affect outcomes significantly. Hence, the described adaptive and personalized setpoint adjustment algorithms described herein achieves better glycemic performance for the user.

The improved glycemic performance may be based on one or more of historical data evaluation and obtaining personalized factors, a determination of a setpoint adjustment based on a combined personalized factor, one or more personalized factors, safety bounds for the new target glucose setpoint, and/or a special occurrence.

The drug delivery system may also include steps where the processor, when adjusting the user's target glucose setpoint is further operable to utilize the personalized factor in a setpoint change calculation. The adjustment to the user's target glucose setpoint may be within a limited range of values based on the personalized factor(s).

The following is a discussion of a determination of a target glucose setpoint adjustment using the user's personalized factor(s). In this adjustment example, the calculated personalized factor is used to adjust the user's target glucose setpoint using the following equation:

$$Setpoint Change ( mg / dL ) = ( 0.5 - P F ) \times α , \qquad ( Equation 3 )$$

where PF is the personalized factor or combined personalized factor and α is an adaptation speed coefficient.

The adaptation speed coefficient α may be set to equal 10 if the user's combined PF indicates the user is advancing towards extreme hypoglycemia (in which case the user's combined PF may be approximately zero, as explained in further detail below). With an adaptation speed coefficient α set equal to 10 and a combined PF determined to be approximately 0, the target glucose setpoint adjustment would be approximately 5 mg/dL. The target glucose setpoint is increased by the processor for hypoglycemic users in order to protect them from further hypoglycemic events. Alternatively, a may also be set to equal 10 when the user's combined PF indicates the user's tends toward extreme hyperglycemia in which case the user's combined PF is approximately one, as explained below. In this case, the target glucose setpoint adjustment will be approximately (−) 5 mg/dL. The target glucose setpoint is decreased by the processor for user's who tend toward hyperglycemia in order to protect them from potential hyperglycemic events.

As for user's whose combined PF is approximately 0.5 (indicating the user is substantially euglycemic), the adaptation speed coefficient α may remain equal to 10. The processor determines the target glucose setpoint adjustment will be ˜0 mg/dL. The target glucose setpoint is maintained at almost the same setting for euglycemic users in order to maintain their nearly-perfect glycemic control outcomes.

The maximum or minimum target glucose setpoint adjustment may be limited as it depends on the adaptation speed coefficient α. The greater the selected value of α, the faster the adaptation of the target glucose setpoint, which in some cases may be unwanted. A lower selected value of the adaptation speed coefficient α results in a slower adaptation, which may be a more optimal approach. Exemplary values of the adaptation speed coefficient α include 1 to 50, or 1 to 30, or 1 to 10, where a higher number causes increased speed of adaptation. One way to control adaptation in addition to, or as an alternative to, changing the adaptation speed coefficient is to limit the target glucose setpoint changes, independently from the selection of the adaptation speed coefficient α.

One proposed process for limiting the adjustment of the set point follows:

$$Setpoint {Change}_{LIMITED} = max ( - 5 , min ( 5 , Setpoint Change ) ) . \qquad ( Equation 4 )$$

Using the max-min function, the MDA may ensure that daily target glucose setpoint adjustments may not be lower than −5 mg/dL and cannot exceed 5 mg/dL. While the values of −5 and 5 are used in the examples, other values may be selected such as −3 and 3 or −8 and 8 as well as differing values such as −5 and 2 or −3 and 6 or the like. In some embodiments, the maximum daily target glucose setpoint adjustment is between about −10 mg/dL to about 10 mg/dL, more specifically between about −7 mg/dL to about 7 mg/dL and in particular between about −5 mg/dL to about 5 mg/dL.

In another example process, the combined personalized factor may be utilized to set constraint boundaries for a new target glucose setpoint for the user. The target glucose setpoint may be set daily or at some other period of time. For example, the process 600 may have be operable to calculate the new target glucose setpoint (also referred to as a new setpoint) based on following equation:

$$New Setpoint = max \qquad ( Equation 5 )$$

$$( 90 , min ( 150 , Previous Day Setpoint + Setpoint Change ) ) ,$$

where Setpoint Change is calculated in Equation 3 and the Previous Day Setpoint is self explanatory.

It is envisioned that the target glucose setpoint dynamically fluctuates between 90-150 mg/dL based on recent glycemic outcomes and evaluations. As such, the new setpoint equation above is formulated to limit the new target glucose setpoint so that it does not exceed 150 mg/dL (upper boundary) and is not lower than 90 mg/dL (lower boundary). Of course, these lower and upper boundaries of 90 mg/dL and 150 mg/dL, respectively, may be set differently to be optimized to a user's sensitivities to these boundaries. For example, a particular user may react differently to a glucose measurement value of 80 mg/dL as compared to another user. As noted with respect to the New Setpoint of Equation 5 above, the target glucose setpoint may be set daily, but the new target glucose setpoint may be updated more frequently, such as every 12 hours, or less frequently, such as every 3 days or the like.

In some instances, there may be special occurrences that may be addressed by the processor, such as, for example, a crashing glucose (i.e., a high rate of negative change of a user's glucose level indicating the potential for, or onset of, a hypoglycemic event), a one-time constraint relaxation, an auto bolus constraint, a hypo-protect mode, or an activity mode.

In a further example, when the following special circumstances occur, the processor may determine whether one or more of the events across the top row occur, and in response, the processor is operable to make a final decision based on the events/modes in Table 1 below.

TABLE 1

| | Normal | | | | |
|---|---|---|---|---|---|
| | Operation | | One- | Auto | |
| | (No | Crashing | Time | Bolus | Hypo |
| | Special | BG | Constraint | Constraint | Protect |
| | Event) | Mode | Relaxation | Relaxation | Mode |
| Final Setpoint | Fuzzy- | User | Fuzzy- | Fuzzy- | Hypo |
| Determination | Logic | Setpoint | Logic | Logic | protect |
| (mg/dL) | Suggestion | (e.g., | Suggestion | Suggestion | overrules, |
| | (e.g., | target | (e.g., | (e.g., | apply |
| | suggestion | glucose | Normal | Normal | hypo |
| | from | setpoint | Operation | Operation | protect |
| | process | set by | plus) + | plus) + | rules |
| | 200) | the user) | 20 mg/dL | 20 mg/dL | |
| | | still | | | |
| | | applies | | | |

For ensuring safety (e.g., not overdelivering or underdelivering a drug dosage), the process 600 may be further operable to cause the processor to set upper and lower boundaries for a meal bolus dose as a percentage (%) of the user's TDI according to the following equation:

$$( x + One Touch Dose Change ) = \qquad ( Equation 6 )$$

$$min ( max ( ( x + One Touch Dose Change ) , 0.02 ) , 0.1 ,$$

where the meal dose % TDI constraint maximum may be set at 0.10 (10%) and a minimum may be set at 0.02 (2%). Other values may be used. In some embodiments, the constraint maximum is between about 0.03 (3%) and about 0.2 (20%), more specifically between about 0.05 (5%) and about 0.13 (13%). In some embodiments, the constraint minimum is between about 0.005 (0.5%) and about 0.08 (8%), more specifically between about 0.01 (0.1%) and about 0.04 (4%).

Based on these exemplary safety constraint values, the meal dose % TDI constraint cannot exceed 10% (i.e., 0.10) and cannot be lower than 2% (i.e., 0.02%), in case the proposed algorithm consistently suggests an increase/decrease of the immediate meal bolus dose. Of course, the upper meal dose % TDI constraint may be greater than or less than 0.10 and the lower meal dose % TDI constraint may be greater than or less than 0.02. Both the maximum meal dose % TDI constraint setting and the minimum meal dose % TDI constraint setting and may be tuned by the processor on a regular basis, such as daily, hourly, at meal times, per operational cycle, or the like. The tuning (up or down) may be based on the user's individual personalized factors or combined personalized factor.

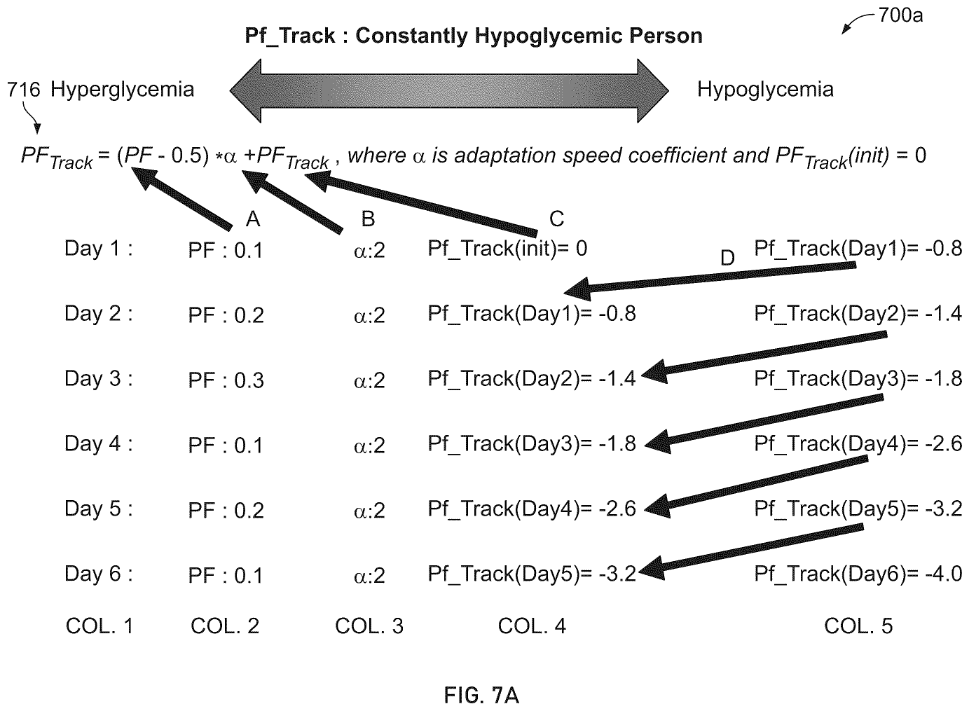

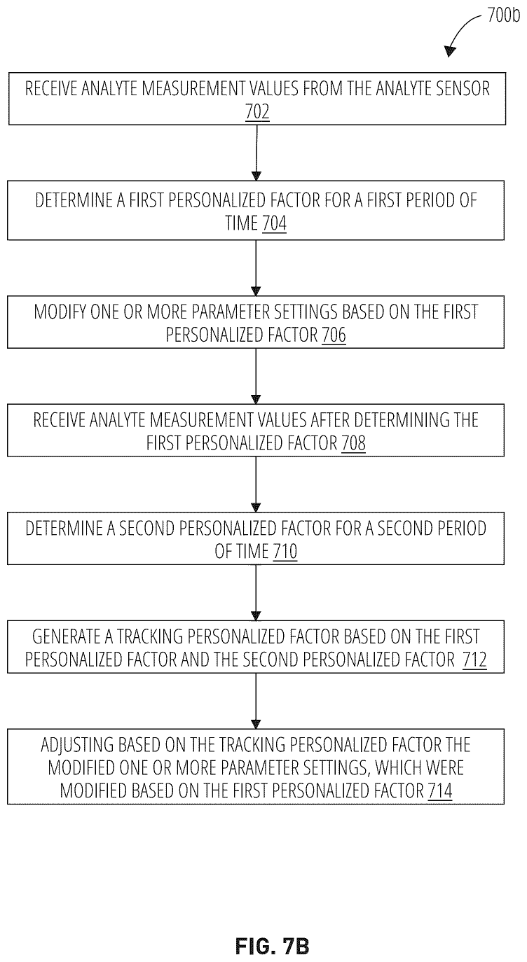

FIG. 7A illustrates an example for tracking a user's combined personalized factor during a period of time in accordance with at least one embodiment of the disclosed subject matter. The MDA may also keep a record of the user's personalized factors and combined personalized factor over a period of time.

An exemplary drug delivery system as described herein is operable to propose aggressive constraint relaxation settings for hyperglycemic users and conservative settings for hypoglycemic users. To do so, the drug delivery system may determine a user's glycemic risk factors (or individual personalized factors) and combine them to determine a combined personalized factor. Since deciding a user's glycemic tendency (toward hypo- or hyper-glycemia) in one day or over a short period of time is challenging, a drug delivery system (shown in a later example) may be operable to track personalized factors or a combined personalized factor over time using, for example, the following:

$$P {F}_{-} Track ( Y ) = ( P F - 0.5 ) * ∝ + P {F}_{-} Track ( init ) , \qquad ( Equation 7 )$$

where ∝ is adaptation speed coefficient, the variable Y is a tunable time period, PF is the user's combined personalized factor derived as explained above, and PF_Track(init)=0. Single personalized factors may also be tracked in this manner. In this example, the value of the adaptation speed coefficient α may be 1.5, 10, a number within 1.0-50, or the like. Accordingly, in some embodiments the adaptation speed coefficient may be between about 0.01 to about 200, more specifically between about 0.5 to about 100 and in particular between about 1.0 to about 50.

In determining the PF_Track(Y), the tunable time period represented by Y may be one day, one week, 24 hours, 36 hours, 72 hours, or the like. Accordingly, in some embodiments the tunable time period Y is between about 1 day to about 1 week, more specifically between about 12 hours to 72 hours.

In this example, the processor may be operable to identify three regions for a PF_Track value, which determine the user's glycemic risk factor based on the following exemplary thresholds:
I. PF Track<−1.5: A Hypoglycemic User
II. PF Track between −1.5 and 1.5: A Euglycemic User
III. PF Track>1.5: A Hyperglycemic User

Note that the value of PF_Track is not limited to a range of 0-1 as may be the case with the combined personalized factor, but is instead limited to the three regions indicated by the ranges: I (e.g., values less than −1.5), II (e.g., values between −1.5 and 1.5, inclusive), and III (e.g., values greater than 1.5). Alternatively, when the PF_Track falls within a range (i.e., I, II or III), the user may be identified by a categorization as a Hypoglycemic User, when the PF_Track is within range I, a Euglycemic user, when the PF_Track value falls with range II, and a Hyperglycemic user, when the PF_Track value falls with range III. In an operational example, the processor, after the determination of user's glycemic risk using the above thresholds, may be operable to adjust the constraint relaxation parameters accordingly. Example settings are illustrated in Table 2 below and are discussed in more depth below.

In the example shown in FIG. 7A, the first column (COL. 1) in the PF Tracking Process 700a is the label of days. Exemplary Day 1 through Day 6 are shown in the example. The second column (COL. 2) is the determined combined personalized factor (PF) for the day (though this could be a single personalized factor). The third column (COL. 3) is the exemplary adaptation speed coefficient ∝ (in this case, 2). The fourth column (COL. 4) is the initial PF_Track value or the previous day's PF_Track value, and the fifth column (COL. 5) is the output of the equation 716 for the indicated day.

In the example PF Tracking Process 700a, in Day 1, the combined personalized factor (PF) is 0.1, and is input into PF Track Equation 716 as PF as indicated by arrow A, the adaptation speed coefficient α is set to 2 and is input as shown by arrow B, and since Day 1 is the initial day, the PF_Track(init) (also referred to as PF_Track(previous day)) is set to 0. The PF_Track(Day 1) value is determined to be (−) 0.8. On Day 1, the processor may evaluate the PF_Track(Day 1) value based on the information shown in Table 2. Since the PF_Track(Day 1) value is (−)0.8 (representing euglycemia or a euglycemic user, according to the chart above), the aggressiveness of the settings for Day 1 are Normal Settings for each of the listed parameters of the MDA, and are not adapted up or down from the Normal Settings. Exemplary Normal Settings are listed in Table 2 below.

The PF_Track(Day 1) value may be stored in drug delivery system memory, a cloud-based service, or both, and may be used on Day 2 to calculate a PF_Track(Day 2) (as shown by arrow D). The Day 2 calculation utilizes the exemplary combined personalized factor (PF) value of 0.2 and the previous day PF_Track(Day 1) value of (−)0.8, which results in a PF_Track(Day 2) value of (−)1.4. The Day 2 PF_Track value of (−)1.4 still falls with the Normal Settings (according to the exemplary thresholds listed above) so the parameter settings for the MDA remain substantially the same as those for Day 1.

At Day 3, however, we begin to note that the PF Tracking Process 700a is beginning to detect a trend in the user's combined PF value (in this case, the PF values are tending toward hypoglcyemia). In the example, this is the third day in a row that the combined PF is on the hypoglycemia side of the range, or close the severe hypoglycemia threshold of 0. In Day 3, the combined personalized factor (PF) is determined to be 0.3, the adaptation speed coefficient remains at the value of 2, and the PF_Track(Day 2) value is (−)1.4. The day 3 PF_Track value is (−)1.8. Since the the PF_Track(Day 3) value is (−)1.8, the aggressiveness of the settings for Day 3 may be set to Conservative Settings for each of the listed parameters of the MDA. Parameters for the remainder of Days 4-6 may remain as Conservative Settings based on the exemplary values calculated in FIG. 4A. Exemplary Conservative Settings, Normal Settings, and Aggressive Settings are depicted in Table 2.

TABLE 2
Example Personalized Constraint Relaxation Settings

| Aggressiveness | Conservative | Normal | Aggressive |
|---|---|---|---|
| of Settings | Settings | Settings | Settings |
| Categorized | PF Track <−1.5 | PF Track >−1.5 | PF Track >1.5 |
| PF Track | (Hypoglycemic | && | (Hyperglycemic |
| | Users) | PF Track <1.5 | Users) |
| | | (Euglycemic Users) | |
| Duration of | 60 | 60 | 60 |
| Setting | | | |
| (Minutes) | | | |
| Set Point | User SP-20 | User SP-20 | User SP-20 |
| During | (Min 90) | (Min 90) | (Min 90) |
| Relaxation | | | |
| One Time | 9× | 12× | 15× |
| Constraint | | | |
| Integral | 5*3 | 5*3 | 5*3 |
| Constraint | | | |
| DOB | 1.5 Times | 1.8 Times | 2.1 Times |
| constraint | (50%) | (80%) | (210%) |
| Zero-Cost | 3× basal | 3× basal | 3× basal |
| Function | | | |
| Region | | | |

Continuing with the operational example, the processes executed by the drug delivery system may also include a step where the processor, prior to retrieving a history of glucose measurement values, receives a request to deliver a bolus dosage and modifies the parameter settings in response to the bolus request.

Alternatively, the processor may be operable to receive a request to deliver a bolus after modifying the parameter setting. The processor may determine a bolus dosage according to the modified parameter setting of the medicament delivery algorithm.

The different constraints listed in Table 2 may be described as follows:

“One time constraint” refers to an amount of drug in Units that can be delivered at one time to a user. The amount of the drug is a factor of the amount of drug delivered via the basal dosage. In an example, if the basal dosage is 2 U per day and the one time constraint is set at 4×U, the medicament delivery algorithm is limited to delivering 4 times 2 U or 8 U per day via basal deliveries. The one time constraint is a tunable parameter. Basal dosages may, for example, be calculated using TDI divided by 48. For example, TDI/48/12=regular basal deliveries per 5-minute cycle, and the one time constraint may be calculated on a per-cycle basis, and exemplary constraint values of 9× or 12× or 15× may be used as shown in Table 2. The length of the cycles may differ. In some embodiments, the cycle length is between about 1 to 15 minutes, more specifically between about 3 minutes to 8 minutes.

“Integral constraint” may refer to a total amount of insulin delivered over a period of time (e.g., Y hours not more than Z times basal may be delivered). This may be applied over a Y hour period and limited to Z times basal. For example, an integral constraint of 15 may be based on a 3 hour period and 5 times basal, resulting in an exemplary per hour delivery limitation of TDI/48×15. As a result, the processor cannot deliver more than the integral constraint within the 3 hour window. In this example, the integral constraint is for basal dosages times 3 hours—which may be a rolling 3-hour window and determined every cycle rather than determined anew every 3 hours. In the example of Table 2, the integral constraint is set to 5 times 3 hours and is also in Units. According to the example of Table 2, the integral constraint is 5 times 3 would be 15 U, representing the maximum amount of drug delivered within the 3-hour window in this example.

“DOB constraint” refers to drug on board (DOB), measurable in Units, and is a measure of how much drug or medicament, such as insulin, is needed to get a user's glucose to satisfy the target glucose setpoint, factoring in an amount of the drug that the user has onboard (e.g., the user's insulin onboard (IOB), or the amount of insulin delivered over the past X hours (e.g., 4 hours) that has not yet acted in the body). With this constraint applied, the MDA cannot deliver more than the DOB constraint.

The “Zero-Cost Function Region” is the function of true basal insulin differentiation between actual basal rate, which also can be called control penalty of the controller, and a predicted basal rate. A low penalty value results in an increase of the responsiveness or aggressiveness of the controller. A high penalty value results in a conservative control action, or decrease in responsiveness of the controller. Therefore, control strategy can be adjusted by changing the zero-cost function formulation. For example, true basal insulin differentiation between 2 times the actual basal rate systems will be more responsive than 1 times the actual basal rate. Therefore, the combined personalization factor may be used to adjust and determine the zero-cost function definition and, respectively, the responsiveness of the controller on a person-to-person basis that results in achieving better individual glycemic outcomes with personalized control action.

The foregoing processes may also be triggered by a meal auto-detection feature of the MDA with no user input in addition to a situation where the user announces a meal. For example, the user may ingest a meal and forget to announce the meal, a meal auto-detection feature of the MDA may note changes in values output by the analyte sensor as correlating to value changes that occur when the user ingests a meal. As a result, the MDA may begin the processes, such as 200 and 600, as described here.

FIG. 7B illustrates an exemplary flowchart of a process for tracking a user's personalized factor during a period of time in accordance with at least one embodiment of the disclosed subject matter. A drug delivery system for implementing the PF Tracking Process 700b may include a processor, an analyte sensor, and a memory. The processor is operable to execute programming instructions and adapt control parameters related to a medicament delivery algorithm. The memory may be operable to store a medicament delivery algorithm and data related to a user's analyte history, and the analyte sensor may be a continuous glucose monitor, and the analyte measurement values may be glucose measurement values.

In the PF Tracking Process 700b, in a step 702, the processor may be operable to receive analyte measurement values from the analyte sensor. A result of the received analyte measurement values is the determination of a first personalized factor.

at step 704, a processor of the drug delivery system may be operable to determine a first combined personalized factor for a first period of time. Based on the first combined personalized factor, the processor at step 704 may determine one or more new parameter settings based on the first combined personalized factor and then modify one or more parameter settings to the new parameter settings. This may represent a first modification of parameter settings. The analyte sensor may generate a number of analyte measurement values that are transmitted from the analyte sensor for receipt by the processor and/or other devices. At step 706, the processor, after determining the first combined personalized factor, receives additional analyte measurement values.

The processor determines a second combined personalized factor for a second period of time (step 708). In an example, the first period of time and the second period of time are the same duration, and may both be a 24 hour, or a 16 hour, or a 36 hour, or a 3 day period of time, or the like.

In step 710, the processor generates a tracking personalized factor based on the first combined personalized factor and the second combined personalized factor.

At step 712, the processor determines one or more new parameter settings, based on the tracking personalized factor, and then adjusts the previously modified one or more parameter settings, which were previously modified based on the first combined personalized factor, to the one or more new parameter settings. The process may continue on, receiving new analyte measurement values, determining subsequent combined personalized factors and tracking personalized factors, and continuing to make adjustments to the one or more parameter settings, thereby adjusting medicament delivery.

In a further operation, the processor may receive a meal announcement, and generate a bolus dosage based on the adjusted, one or more modified parameter settings for delivery from a wearable medicament delivery device. Other technical features may be readily apparent to one skilled in the art from the following figures, descriptions, and claims.

FIG. 8 illustrates a simplified block diagram of a drug delivery system 800. The drug delivery system 800 may include a controller 802, a wearable medicament delivery device 804, an analyte sensor(s) 806, a network 808, and cloud-based service(s) 850. The controller 802 may also include communication device that may have a transceiver 810 and a transceiver 812, a user interface 814, a processor 816, and a memory 818. The memory 818 may include other data 820, a control application 822, and settings 824. The wearable medicament delivery device 804 may include a communication device 826, a reservoir 828, a memory 830, a pump 832, a user interface 834, and a processor 836. The memory 830 may include settings 838, a control application 840, and other data 842. The system 800 may also include a fitness device 844, a smart accessory device 846, other wearable device(s) 848, as well as communication link 852, communication link 854, communication links 858, and a computing device 856. Details of the respective devices are described in further depth below.

The wearable medicament delivery device 804 may deliver medicament such as drugs, medications, or therapeutic agents suitable for automated delivery, such as insulin, morphine, methadone, hormones, fertility drugs, blood pressure medicines, chemotherapy drugs, weight loss drugs, glucagon, glucagon-like peptide-1 receptor agonist (GLP-1), glucose-dependent insulinotropic polypeptide (GIP), and/or combinations of medicaments, such as insulin and (GLP-1) and/or GIP, or the like.

In some examples, the drug delivery system 800 is suitable for delivering insulin to a user in accordance with the disclosed embodiments. The drug delivery system 800 may include a wearable medicament delivery device 804, a controller 802, and an analyte sensor(s) 806. In addition, the drug delivery system may interact with a computing device 856 via Network 808 as well as obtain or contribute to cloud-based service(s) 850.

The wearable medicament delivery device 804 may be a wearable device that is worn on the body of the user. The wearable medicament delivery device 804 may be directly coupled to a user (e.g., directly attached to the skin of the user via an adhesive, or the like at various locations on the user's body, such as thigh, abdomen, or upper arm). In an example, a surface of the wearable medicament delivery device 804 may include an adhesive to facilitate attachment to the skin of a user.

The wearable medicament delivery device 804 may include a processor 836. The processor 836 may be implemented in hardware, software, or any combination thereof. The processor 836 may, for example, be a microprocessor, a logic circuit, a field programmable gate array (FPGA), an application specific integrated circuit (ASIC) or a microprocessor coupled to a memory. The processor 836 may maintain a date and time as well as be operable to perform other functions (e.g., calculations or the like). The processor 836 may be operable to execute programming instructions related to the different functions performed by the wearable medicament delivery device 804. The programming instructions may include a control application 840 stored in the memory 830 that enables the processor 836 to direct operation of the wearable medicament delivery device 804. The control application 840 may control medicament delivery to the user per an MDA. The memory 830 may store control application 840 settings for a user, such as the different constraints of Table 2, MDA settings, such as maximum medicament delivery, medicament sensitivity settings, total daily medicament settings, such as TDI, and the like.

The analyte sensor(s) 806 may be operable to collect physiological condition data, such as the glucose measurement value and a timestamp, ketone levels, heart rate, blood oxygen levels and the like that may be shared with the wearable medicament delivery device 504, the controller 802 or both. For example, the communication device 826 of the wearable medicament delivery device 804 may be operable to communicate with the analyte sensor(s) 806 and the controller 802 as well as the devices 844, 846 and 848. The communication device 826 of the wearable medicament delivery device 804 includes communication circuitry, such as a transceiver, transmitter or receiver, that may be operable to communicate via one or more of Bluetooth®, Wi-Fi, a near-field communication standard, a cellular standard, or any other wireless protocol.

The user interface 834 may have one or more of a microphone, a speaker, a vibration device, a display, a push button, a touchscreen display, a tactile input surface, or the like. The user interface 834 may be coupled to the processor 836 and may include circuitry operable to generate signals based on received inputs and provide the generated signals to the processor 836.

The wearable medicament delivery device 804 may include a reservoir 828. The reservoir 828 may be operable to store medicament suitable for automated delivery as described above. A fluid path to the user may be provided via tubing and a needle/cannula (not shown). The fluid path may, for example, include tubing coupling the wearable medicament delivery device 804 to the user (e.g., via tubing coupling a needle or cannula to the reservoir 828). The wearable medicament delivery device 804 may be operable based on control signals from the processor 836 to expel the drugs, medications or therapeutic agents, such as insulin, from the reservoir 828 to deliver doses of the drugs, medications or therapeutic agents, such as the insulin, to the user via the fluid path. For example, the processor 836 by sending control signals to the pump 832 may be operable to cause insulin to be expelled from the reservoir 828.

There may be one or more communication links 858 with one or more devices physically separated from the wearable medicament delivery device 804 including, for example, a controller 802 of the user and/or a caregiver of the user and/or an analyte sensor(s) 806. The analyte sensor(s) 806 may communicate with the wearable medicament delivery device 804 via a wireless communication link 854 and/or may communicate with the controller 802 via a wireless communication link 852. The communication links 854 and 552 may include wired or wireless communication paths operating according to any known communications protocol or standard, such as Bluetooth, Wi-Fi, a near-field communication standard, a cellular standard, or any other wireless protocol.

The wearable medicament delivery device 804 may also include a user interface (UI) user interface 834, such as an integrated display device for displaying information to the user, and in some embodiments, receiving information from the user. For example, the user interface 834 may include a touchscreen and/or one or more input devices, such as buttons, knob or a keyboard that enable a user to provide an input.

In addition, the processor 836 may be operable to receive data or information from the analyte sensor analyte sensor(s) 806 as well as other devices, such as smart accessory device 846, fitness device 844, and/or other wearable device(s) 848 (e.g., a blood oxygen sensor or the like), that may be operable to communicate with the wearable medicament delivery device 804. For example, fitness device 844 may include a heart rate sensor and be operable to provide heart rate information, or the like.

The wearable medicament delivery device 804 may interface with a Network 808. The Network 808 may include a local area network (LAN), a wide area network (WAN), a cellular network, or a combination thereof and operable to be coupled wirelessly to the wearable medicament delivery device 504, the controller 802, and devices 544, 546, and 548. A computing device 856 may be interfaced with the Network 808, and the computing device 856 may communicate with the wearable medicament delivery device 804. The computing device 856 may be a healthcare provider device, a guardian's computing device, or the like through which a user's controller 802 may interact to obtain information, store settings, and the like. The control application 822 may be operable to execute an MDA and present a graphical user interface on the computing device 856 enabling the input and presentation of information related to the MDA.

The drug delivery system 800 may include an analyte sensor(s) 806 for detecting the analyte levels of one or more analytes of a user, such as glucose levels, ketone levels, other analytes relevant to an illness treatment program, such as diabetes or chemotherapy regimen, or the like. The analyte level values detected may be used as physiological condition data and be sent to the controller 802 and/or the wearable medicament delivery device 804. The analyte sensor(s) 806 may be coupled to the user by, for example, adhesives or the like and may provide information or data on one or more medical conditions and/or physical attributes of the user. The analyte sensor(s) 806 may be a continuous glucose monitor (CGM), ketone sensor, or another type of device or sensor that provides glucose measurements that is operable to provide glucose concentration measurements. The analyte sensor(s) 806 may be physically separate from the wearable medicament delivery device 804 or may be an integrated component thereof. The analyte sensor(s) 806 may provide the processor 836 and/or processor 816 with physiological condition data indicative of measured or detected glucose levels of the user. The information or data provided by the analyte sensor(s) 806 may be used to modify an insulin delivery schedule and thereby cause the adjustment of drug delivery operations of the wearable medicament delivery device 804.

In the depicted example, the controller 802 may include a processor 816 and a memory 818. The controller 802 may be a special purpose device, such as a dedicated personal diabetes manager (PDM) device. The controller 802 may be a programmed general-purpose device that is a portable electronic device, such as any portable electronic device, smartphone, smartwatch, fitness device, tablet or the like including, for example, a dedicated processor, such as processor, a micro-processor or the like. The controller 802 may be used to program or adjust operation of the wearable medicament delivery device 804 and/or the analyte sensor(s) 806. The processor 816 may execute processes to manage a user's glucose levels and control the delivery of the drug or a therapeutic agent (e.g., a liquid drug or the like as mentioned above) to the user. The processor 816 may also be operable to execute programming code stored in the memory 818. For example, the memory 818 may be operable to store a control application 822 for execution by the processor 816. The control application 822 may be responsible for controlling the wearable medicament delivery device 804, including the automatic delivery of insulin based on recommendations and instructions from the MDA, such as those recommendations and instructions described herein.

The memory 818 may store one or more applications, such as the control application 822, which may be the same as, or substantially the same as those described above with reference to the wearable medicament delivery device 804. In addition, the settings 838 may store information, such as drug delivery history, glucose measurement value over a period of time, total daily insulin values, and the like. The memory 818 may be further operable to store as other data 820 data, such as analyte history (e.g., analyte measurement values over time), MDA settings and parameters, medicament treatment program history (such as insulin delivery history, glucose measurement value history, and the like) as well as drug-on-board information, such as IOB, or insulin-to-carbohydrate ratio (ICR) or the like.

The controller 802 may include a user interface (UI) 814 for communicating visually with the user. The user interface 814 may include a display, such as a touchscreen, for displaying information provided by the control application 822. The touchscreen may also be used to receive input when it is a touch screen. The user interface 814 may also include input elements, such as a keyboard, button, knob or the like. In an operational example, the user interface 814 may include a touchscreen display controllable by the processor 816 and be operable to present the graphical user interface, and in response to a received input (audio or tactile), the touchscreen display is operable present a graphical user interface related to the received input.

The controller 802 may interface via a wireless communication link of the wireless communication links 198 with a network, such as a LAN or WAN or cellular network or combination of such networks that provides one or more servers or cloud-based service(s) 850 via communication device 860. The communication device 860, which may include transceivers 810 and 812, may be coupled to the processor 816. The communication device 860 may be operable to transmit communication signals (e.g., command and control signals) to and receive communication signals (e.g., via transceivers 810 and/or 512) from the wearable medicament delivery device 804 and the analyte sensor analyte sensor(s) 806. In an example, the transceiver 810 may a first transceiver and may be a Bluetooth transceiver, which is operable to communicate with the communication device 826 of the wearable medicament delivery device 804, and the transceiver 812 may be a second transceiver that may be a cellular transceiver, a Bluetooth® transceiver, a near-field communication transceiver, or a Wi-Fi transceiver operable to communicate via the Network 808 with computing device 856 or with cloud-based service(s) 850. While two transceivers 810 and 812 are shown, it is envisioned that the controller 802 may be equipped with a number of transceivers, such as a cellular transceiver, a Bluetooth transceiver, a near-field communication transceiver, or a Wi-Fi transceiver.

Other devices, like smart accessory device 846 (e.g., a smartwatch or the like), fitness device 844 and other wearable device other wearable device(s) 848 may be part of the drug delivery system 800. These devices may communicate with the wearable medicament delivery device 804 to receive information and/or issue commands to the wearable medicament delivery device 804. These devices 544, 846, and 848 may execute computer programming instructions to perform some of the control functions otherwise performed by processor 816 or processor 836. These devices 844, 846, and 848 may include user interfaces, such as touchscreen displays for displaying information such as current glucose level, insulin on board, analyte histories, insulin deliver history, or other parameters or treatment-related information and/or receiving inputs. The display may, for example, be operable to present a graphical user interface for providing input, such as request a change in basal insulin dosage or delivery of a bolus of insulin. Devices 844, 846, and 848 may also have wireless communication connections with the analyte sensor(s) 806 to directly receive analyte measurement values and level data as well as other data, such as user history data maintained by the controller 802 and/or the wearable medicament delivery device 804.

The user interface 834 may be a touchscreen display controlled by the processor 836, and the user interface 834 is operable to present a graphical user interface that offers an input of a subjective insulin need parameter usable by the control application 840. The processor 836 may cause a graphical user interface to be presented on the user interface 834. The control application 840 may generate instructions for the pump 832 to deliver basal insulin to the user or the like.

As described with reference to earlier examples, the processor 836 and/or processor 816 may be operable to perform calculations regarding settings of the MDA as discussed as herein. The modifications to the MDA settings may be stored in the memory 830 or memory 818.

The drug delivery system 500 may communicate with or receive services from a cloud server providing cloud-based service(s) 850. A cloud server may be configured to provide one or more of the cloud-based service(s) 850. The cloud-based service(s) 850 may include data storage that stores personal or anonymized data, such as glucose measurement values, historical IOB or TDI, prior day personalized factors, parameter settings as well as modified parameter settings, prior carbohydrate-compensation dosages, and/or other forms of data. In addition, the cloud-based service(s) 850 may process anonymized data from multiple users to provide generalized information related to TDI, insulin sensitivity, IOB and the like. The communication link 862 that couples the cloud-based service(s) 850 to other components of drug delivery system 800, for example, controller 802, wearable medicament delivery device 804, analyte sensor(s) 806 and the like may be a cellular communication link, a Wi-Fi link, a Bluetooth® link, other respective wireless protocol, or a combination thereof.

While the implementation of a cost function is described at a high level herein additional details may be provided as described in Applicant's U.S. patent application Ser. Nos. 17/330,115 and 17/534,129, the entire contents of which are incorporated herein by reference.

Software related implementations of the techniques described herein, such as the examples described with reference to FIGS. 1-16 may include, but are not limited to, firmware, application specific software, applet, or any other type of computer readable instructions that may be executed by one or more processors. The computer readable instructions may be provided via non-transitory computer-readable media. The various elements of the processes described with reference to the figures may be implemented in devices, apparatuses or systems that may include various hardware elements, software elements, or a combination of both. Examples of hardware elements that may be the basis for a computer processor may include structural members, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, processes, procedures, software interfaces, application program interfaces (API), instruction sets, computing code, computer code, code segments, computer code segments, words, values, symbols, or any combination thereof.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined solely by the preceding illustrative description.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

1. A drug delivery system, comprising:
a memory configured to store programming code and data;
a processor coupled to the memory and operable to execute the programming code, wherein the processor, when executing the programming code, is operable to:
receive a plurality of glucose measurement value;
extract a plurality of feature values from the received plurality of glucose measurement value;
assign a value to each respective extracted feature value of a plurality of extracted feature values to generate respective weighted extracted feature values;
calculating a combined personalized factor based on the respective weighting of each of the plurality of extracted feature values; and
utilize the combined personalized factor to calculate a medicament dosage to be delivered to a user.

2. The system of claim 1, wherein when calculating the personalized factor, the processor is operable to:
determine a degree to which the respective weighted extracted feature values indicate a user bias toward hyperglycemia or hypoglycemia; and
assign a value as the combined personalized factor based on the determined degree.

3. The system of claim 2, wherein the calculated personalized factor has a value that falls on a scale between severe hyperglycemia and severe hypoglycemia.

4. The system of claim 1, wherein the processor, when calculating the combined personalized factor, is further operable to:
apply a weighting algorithm to the plurality of extracted feature values, wherein each extracted feature value has a respective value;
obtain from the weighting algorithm a respective weight value assigned to each of the plurality of extracted feature values;
apply the respective weight value assigned to each respective extracted feature value of the plurality of extracted feature values;
normalize each respective weighted extracted feature values;
determine an average value of the normalized weighted extracted feature values; and
set the average value as the combined personalized factor.

5. A drug delivery system, comprising:
a processor; and
a memory operable to store a medicament delivery algorithm that, when executed by the processor, cause the processor to:
receive a meal announcement;
retrieve a history of glucose measurement values;
extract feature values from the history of glucose measurement values;
determine a weighted extracted feature value by applying a weight value to each respective extracted feature value;
determine a personalized factor based on the weighted extracted feature value; and
based on the personalized factor, modify a parameter setting of the medicament delivery algorithm.

6. The drug delivery system of claim 5, wherein the processor is further operable to:
determine a bolus dosage amount based on the modified parameter setting of the medicament delivery algorithm.

7. The drug delivery system of claim 5, wherein the modified parameter setting is at least one or more of target glucose set point, a one time constraint, an integral constraint, a DOB constraint or a cost function multiplier.

8. The drug delivery system of claim 5, wherein the processor, prior to applying the weight value to each respective extracted feature value, is operable to:
input the extracted feature value into a weighting algorithm, wherein the weighting algorithm is configured to determine a respective weight value for the applied weighting.

9. The drug delivery system of claim 5, wherein the processor, when determining the personalized factor, is operable to:
evaluate the weighted extracted feature value utilizing a rule-based methodology; and
output the personalized factor.

10. The drug delivery system of claim 5, wherein the personalized factor is an indicator of a user's glycemic risk.

11. The drug delivery system of claim 5, wherein the processor when modifying the parameter setting is operable to:
adjust a user's target glucose setpoint.

12. The drug delivery system of claim 11, wherein the processor, when adjusting the user's target glucose setpoint, is further operable to:
utilize the personalized factor in a setpoint change calculation, wherein the adjustment to the user's target glucose setpoint is within a limited range of values based on the personalized factor.

13. The drug delivery system of claim 5, wherein the processor, prior to retrieving the history of glucose measurement values is operable to:
receive a request to deliver a bolus dosage.

14. The drug delivery system of claim 5, wherein the processor is further operable to:
receive a request to deliver a bolus; and
determine a bolus dosage according to the modified parameter setting of the medicament delivery algorithm.

15. A drug delivery system, comprising:
a processor operable to execute programming instructions related to a medicament delivery algorithm;
a memory operable to store the medicament delivery algorithm and data related to a user's analyte history, wherein the processor is operable to:
determine a first personalized factor for a first period of time;
modify one or more parameter settings based on the first personalized factor;
receive analyte measurement values after determining the first personalized factor;
determine a second personalized factor for a second period of time based on the received analyte measurement values;
generate a tracking personalized factor based on the first personalized factor and the second personalized factor; and
adjust based on the tracking personalized factor the modified one or more parameter settings, which were modified based on the first personalized factor.

16. The drug delivery system of claim 15, wherein the first period of time and the second period of time are both a 24 hour period of time.

17. The drug delivery system of claim 15, further comprising:
an analyte sensor operable to generate analyte measurement values from the user, wherein the analyte history includes analyte measurement values generated by the analyte sensor.

18. The drug delivery system of claim 17, wherein the analyte sensor is a continuous glucose monitor, and
the analyte measurement values are glucose measurement values, which are stored as the analyte history in the memory.

19. The drug delivery system of claim 15, wherein the processor is further operable to:
receive a meal announcement; and
generate a bolus dosage based on the adjusted, one or more modified parameter settings for delivery from a wearable drug delivery device.

20. The drug delivery system of claim 15, wherein the processor is further operable to:
receive a request to deliver a bolus; and
determine a bolus dosage according to the adjusted, one or more modified parameter setting of the medicament delivery algorithm.
